(19) United States
(12) Patent Application Publication
(10) Pub. No.: US 2021/0282642 A1
(43) Pub. Date: Sep. 16, 2021

(54) SCANNING MECHANISM FOR MULTIMODALITY INTRAVASCULAR AND ENDOSCOPIC IMAGING CATHETERS

(71) Applicant: THE REGENTS OF THE UNIVERSITY OF CALIFORNIA, Oakland, CA (US)

(72) Inventors: Zhongping Chen, Irvine, CA (US); Jason Chen, Irvine, CA (US); Yan Li, Irvine, CA (US)

(21) Appl. No.: 17/197,897

(22) Filed: Mar. 10, 2021

Related U.S. Application Data
(60) Provisional application No. 62/988,137, filed on Mar. 11, 2020.

(51) Int. Cl. A61B 5/00

(52) U.S. Cl. CPC A61B 5/0035; A61B 5/0066; A61B 5/7225; A61B 5/0075; A61B 5/7257; A61B 5/6852

(57) ABSTRACT

The present invention is directed to a system for multimodal imaging through the use of a dual-rotational imaging catheter. The system may comprise a swept-source laser for providing a light source for OCT and OCE imaging, and an optical fiber coupler that splits said light source into one for a compensation arm and the other for the imaging catheter. The imaging catheter may comprise a rotary apparatus for a first scanning method, and a distal motor for a second scanning method. The dual-rotational model may allow for optimal performance of multiple imaging modalities. The imaging catheter may utilize optical imaging and acoustic imaging. A balanced photodetector receives input from the destinations of both light sources to offset DC noise. An US pulser/receiver is used for US imaging, a multifunction I/O module, a function generator, and an amplifier are used for generating an acoustic excitation force for OCE imaging.

### CROSS-REFERENCES TO RELATED APPLICATIONS

This application is a non-provisional and claims benefit of U.S. Provisional Application No. 62/988,137 filed Mar. 11, 2020, the specification of which is incorporated herein in its entirety by reference.

### STATEMENT REGARDING FEDERALLY SPONSORED RESEARCH OR DEVELOPMENT

This invention was made with government support under Grant No. R01HL125084 and Grant No. R01HL127271 both awarded by the National Institutes of Health. The government has certain rights in the invention.

### FIELD OF THE INVENTION

The present invention is directed to multimodal imaging utilizing a dual-rotation catheter design to perform precision measurements on tissue for the detection of diseases.

### BACKGROUND OF THE INVENTION

Clinically, early detection of the latent vulnerability of plaques is the first line of defense against the lethal consequences of acute coronary events, and accurate characterization of a plaque lesion can facilitate better treatment management by furthering our understanding in the disease progression. Studies have demonstrated the increased strain in fatty tissue compared to fibrous plaques, as well as the altered strain in vulnerable plaques compared to surrounding tissue. Additionally, the risk of plaque rupturing is related to the stress on the fibrous cap and the plaques composition. Although quantifying the biomechanical properties of artery tissue in vivo is of crucial importance for early diagnosis and disease management of vulnerable plaques, current intravascular imaging techniques, such as ultrasound (US), optical coherence tomography (OCT), and near-infrared spectroscopy (NIRS), are not capable of doing so.

In clinical practice, computed tomography angiography is routinely performed to identify the stenotic region caused by plaque formation via visualization of coronary arteries in two-dimensions, although it lacks the spatial resolution to resolve tissue-level information of the arterial wall, hence the inadequacy in studying vulnerable plaques. The development of modern techniques aims to address this limitation. Intravascular ultrasound (IVUS) and intravascular optical coherence tomography (IVOCT) are currently the most significant clinical adaptations. The main advantage of IVUS and IVOCT lies in their capabilities of providing cross-sectional information of the arterial wall to reveal the underlying layered structure of the vascular tissue. The large penetration depth of IVUS enables the full-depth visualization of the coronary lumen, blood vessel wall, and atherosclerotic plaque formation, and therefore has been routinely utilized in clinical practices. Benefiting from its micron-scale resolution, IVOCT has been proven as a sensitive method for measuring fibrous cap thickness. Nevertheless, IVOCT suffers from shallow penetration depth and cannot completely visualize larger plaques, and IVUS lacks the necessary resolution for microstructure identification. In addition, both IVUS and IVOCT have limited sensitivity for studying chemical composition and quantifying biomechanical properties.

In recent years, several other methods have been explored for assessing plaque in the chemical and biomechanical domains. Intravascular near-infrared fluorescence or spectroscopy (NIRS or NIRS) is capable of providing molecular contrast with high sensitivity for characterizing the intra-lesion lipid content, but its depth information is lacking, hence the limited capability in plaque characterization. Intravascular photoacoustic (IVPA) is based on tissue absorption contrast and has the ability to visualize depth-resolved composition of atherosclerotic plaque; however, it lacks the sensitivity for biomechanical properties. Intravascular optical coherence elastography (IVOCE) is a functional extension of IVOCT, and it allows for point-by-point mapping of arterial wall elasticity by measuring the localized tissue displacement with sub-micrometer/nanometer detection sensitivity. In addition, the plaque type can be identified based on the composition-dependent biomechanical property. Presently, since no single technique can provide a complete assessment of the plaque, several imaging methods are often performed in sequence to achieve a comprehensive evaluation. While the sequential imaging approach can compensate for the limitations of each individual technique, the increased X-ray exposure, procedure length, and associated risks cannot be overlooked. In addition, since data acquisition is performed individually, image co-registration is necessary, which is typically performed off-line manually or semi-automatically. Not only is image co-registration a tedious and time-consuming task, it also has limited accuracy due to human error and interobserver variances. Therefore, a technique that can simultaneously perform multiple imaging technologies through a single intravascular imaging catheter may greatly improve clinical outcomes in cardiology. Although intravascular probing techniques such as integrated US-NIRS, US-OCT, OCT-N IRS, and optical coherence tomography-optical coherence elastography (OCT-OCE) have been recently proposed to facilitate vulnerable plaque diagnostics, they still lack the ability to thoroughly identify all the main characteristics. A OCE system can resolve a localized displacement in the subnanometer range and is therefore ideal for studying the elasticity of biological tissue.

The integration of multiple imaging techniques into one single intravascular probe that is capable of simultaneous acquisition of different tissue characteristics—including structural morphology, chemical composition, and functional elasticity—may lead to a safer, more efficient, and more comprehensive means for plaque characterization. However, the required rotational speed for each imaging modality may differ, and scanning of a multimodal imaging catheter using one speed may be insufficient in obtaining the optimal imaging results from each and all of the modalities within the catheter. In addition to interventional cardiology, other medical specialties relying on endoscopy face similar challenges. Herein, the present invention, while related to intravascular applications, is applicable for endoscopic applications.

### BRIEF SUMMARY OF THE INVENTION

It is an objective of the present invention to provide devices and systems that allow for multimodal imaging utilizing a dual-rotational imaging catheter to allow for multiple simultaneous and optimal scanning methods at one time, as specified in the independent claims. Embodiments of the invention are given in the dependent claims. Embodiments of the present invention can be freely combined with each other if they are not mutually exclusive.

The present invention is an imaging catheter comprising a dual-rotation scanning mechanism that two scanners operate simultaneously but independently. The two scanners may also be operated in a synchronized manner. The scanning apparatus of the catheter comprises two rotary engines, which may be, as a nonlimiting example, a proximal scanning technique such as an optical rotary joint and/or a slip ring driven by an electric motor and a distal scanning technique such as a scanning mirror driven by a micromotor, for providing the operational principle of scanning.

The present invention also includes a method for performing multimodal imaging via the integration of multiple imaging modalities, which may be, for example, fluorescence-lifetime imaging microscopy (FLIM), optical coherence tomography (OCT), optical coherence elastography (OCE), near-infrared fluorescence or spectroscopy (NIRF or NIRS), photoacoustic tomography (PAT), or ultrasound (US). Through the dual-rotation scanning mechanism, the imaging speed (i.e., the rate of which an imaging frame is acquired through a cycle of a scanning mechanism) of each modality can be optimized.

A main object of the invention is to achieve an optimal imaging speed for each imaging modality within a multimodality imaging catheter. Owing to the fact that imaging modalities, such as the aforementioned ones, are based on different physical and biological principles, which may require different imaging speeds for the optimal results, the use of a universal imaging speed that is designed for one modality but is not suitable for other modality or modalities substantially reduces the resulting image quality of a multimodal imaging system. Here, one can take advantage of the dual-rotation scanning mechanism to simultaneously operate two individually controlled scanning principles within an imaging catheter to achieve two imaging speeds, each for an imaging modality or modalities.

Another key advantage of the invention is the increase in the maximal speed of each modality can be achieved. An exemplary case is an intravascular OCT-US dual-modality imaging catheter: if using a conventional single-rotation scanning mechanism, the maximal imaging speed (i.e., the rotational speed of the imaging catheter) is limited by US due to the US acoustic wave which travels at a much slower velocity than the OCT electromagnetic wave. By tuning the rotational speeds individually in a dual-rotation scanning mechanism, the maximal imaging speeds of both US and OCT can be achieved.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The patent application or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1A shows a typical imaging catheter utilizing the proximal scanning approach.

FIG. 1B shows a typical imaging catheter utilizing the distal scanning approach.

FIG. 2A shows an embodiment of an imaging catheter with a dual-rotation scanning mechanism and an acoustic element disposed at the proximal end of an optical assembly.

FIG. 2B shows an embodiment of an imaging catheter with a dual-rotation scanning mechanism and an acoustic element disposed at a distal tip of the imaging catheter.

FIG. 3A shows an embodiment of an imaging catheter with a dual-rotation scanning mechanism and two acoustic elements disposed diametrically at a proximal end of an optical assembly.

FIG. 3B shows an embodiment of an imaging catheter with a dual-rotation scanning mechanism, a first acoustic element disposed at a proximal end of an optical assembly, and a second acoustic element disposed at a distal tip of the imaging catheter.

FIG. 4A shows an embodiment of an imaging catheter with a dual-rotation scanning mechanism, a ring acoustic element, and a rectangle acoustic element disposed at a proximal end of an optical assembly.

FIG. 4B shows an embodiment of an imaging catheter with a dual-rotation scanning mechanism, a ring acoustic element, and a rectangle acoustic element disposed at a distal tip of the imaging catheter.

FIG. 5 shows a schematic of a rotary apparatus on a translational stage, upon which an imaging catheter may be disposed such that said catheter may extend, retract, and rotate.

FIG. 6 shows a schematic of a system for multimodal imaging that utilizes a dual-rotational scanning mechanism in an imaging catheter.

FIG. 7 shows an embodiment of an imaging catheter with a dual-rotation mechanism meant for use in the system of FIG. 6.

FIG. 8 shows a schematic of an alternate system for multimodal imaging that utilizes a dual-rotational scanning mechanism in an imaging catheter.

FIG. 9A shows a 1.3-μm OCT image of a healthy cadaver aorta.

FIG. 9B shows a 1.7-μm OCT image of a healthy cadaver aorta.

FIG. 9C shows a graph of quantitative analysis of penetration depth at the dotted lines in FIG. 9A and FIG. 9B.

FIG. 9D shows a 1.3-μm OCT image of atherosclerotic plaque from a cadaver aorta.

FIG. 9E shows a 1.7-μm OCT image of atherosclerotic plaque from a cadaver aorta.

FIG. 9F shows the histology corresponding to FIG. 13D and FIG. 13E.

FIG. 10A shows an OCT image of a plaque from an ex vivo elastic wave measurement of cadaver arteries.

FIG. 10B shows a time-lapse Doppler image of a plaque from an ex vivo elastic wave measurement of cadaver arteries at 0.0 ms.

FIG. 10C shows a time-lapse Doppler image of a plaque from an ex vivo elastic wave measurement of cadaver arteries at 0.06 ms.

FIG. 10D shows a time-lapse Doppler image of a plaque from an ex vivo elastic wave measurement of cadaver arteries at 0.12 ms.

FIG. 10E shows an H&E histology at the red box region of FIG. 10A.

FIG. 10F shows an OCT image of a normal vascular tissue from an ex vivo elastic wave measurement of cadaver arteries.

FIG. 10G shows a time-lapse Doppler image of a normal vascular artery from an ex vivo elastic wave measurement of cadaver arteries at 0.0 ms.

FIG. 10H shows a time-lapse Doppler image of a normal vascular artery from an ex vivo elastic wave measurement of cadaver arteries at 0.06 ms.

FIG. 10I shows a time-lapse Doppler image of a normal vascular artery from an ex vivo elastic wave measurement of cadaver arteries at 0.12 ms.

FIG. 10J shows an H&E histology at the red box region of FIG. 10F.

FIG. 10K shows a spatiotemporal map at the green dotted line in FIG. 10A.

FIG. 10L shows a spatiotemporal map at the green dotted line in FIG. 10F.

FIG. 11A shows a schematic illustration of a dual-rotational imaging catheter giving excitation force to the blood vessel wall.

FIG. 11B shows a schematic illustration of shear wave propagation along the longitudinal direction of the blood vessel wall.

FIG. 11C shows a schematic illustration of shear wave propagation along the circumferential direction of the blood vessel wall.

FIG. 11D illustrates the wavefront propagation of the shear wave induced by the two diametrically excitation forces in cross-section.

FIG. 12A shows an OCE image of a conventional configuration.

FIG. 12B shows an OCE image of a common-path demonstrating improved phase stability.

FIG. 13 shows schematics for scanning protocols.

FIG. 14 shows the signal processing procedure.

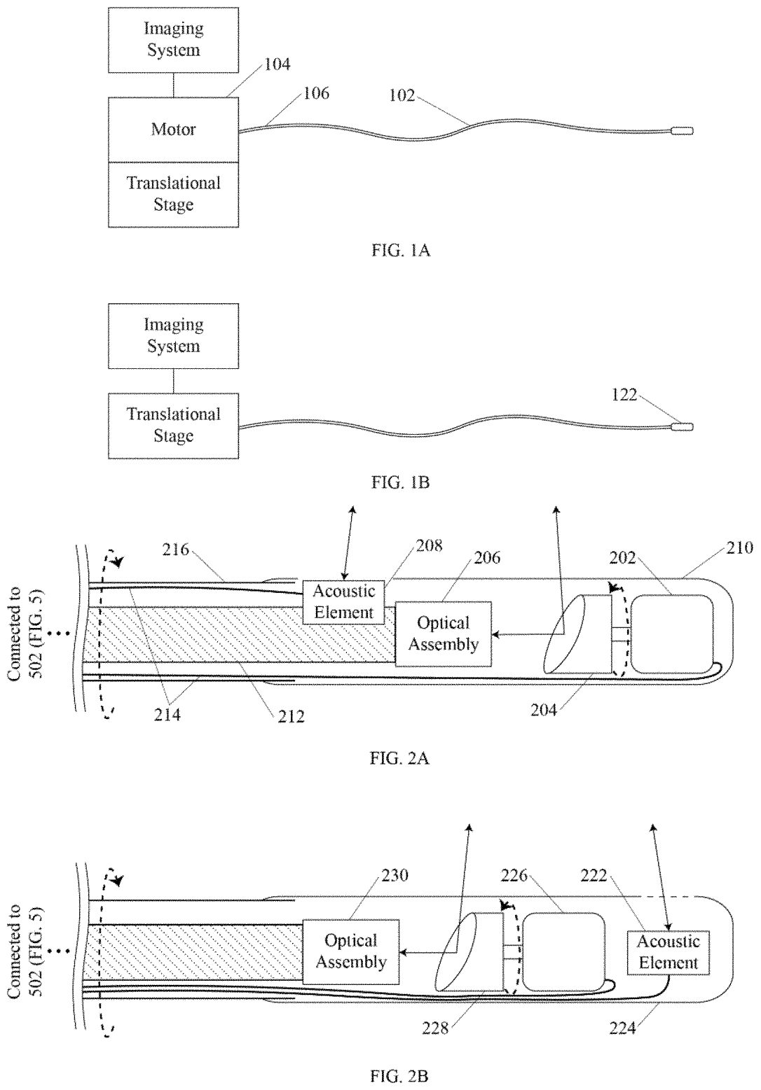

### DETAILED DESCRIPTION OF THE INVENTION

Following is a list of elements corresponding to a particular element referred to herein:
102 shaft of imaging catheter
104 second motor
106 proximal end of shaft
122 distal tip of imaging catheter encapsulating the first motor (202)
202 first motor
204 optically reflective element
206 optical assembly
208 acoustic elements
210 cap for housing distal tip of imaging catheter
212 optical fiber
214 electrical wires for first motor
216 torque coil
222 acoustic element housed distally in cap
224 cap
226 first motor in imaging catheter with acoustic element housed distally in cap
228 optically reflective element in imaging catheter with acoustic element housed distally in cap
230 optical assembly in imaging catheter with acoustic element housed distally in cap
302 first motor in imaging catheter with multiple acoustic elements
304 optically reflective element in imaging catheter with multiple acoustic elements
306 cap
308 optical assembly in imaging catheter with multiple acoustic elements
310 first acoustic element in imaging catheter with multiple acoustic elements
312 second acoustic element in imaging catheter with multiple acoustic elements
322 first acoustic element housed distally in cap in imaging catheter with multiple acoustic elements
324 first motor in imaging catheter with multiple acoustic elements and one housed distally in cap
328 second acoustic element in imaging catheter with multiple acoustic elements and one housed distally in cap
330 optical assembly in imaging catheter with multiple acoustic elements and one housed distally in cap
402 ring acoustic element
404 optical assembly in imaging catheter with ring acoustic element
406 optically and acoustically reflective element in imaging catheter with ring acoustic element
408 first motor in imaging catheter with ring acoustic element
410 acoustic element in imaging catheter with ring acoustic element
422 first motor in imaging catheter with one ring acoustic element and one acoustic element housed distally in cap
424 rectangular acoustic element housed distally in cap in imaging catheter with ring acoustic element
502 rotary apparatus
504 hollow shaft slip ring
506 optical fiber passing through slip ring
508 first connector
510 second connector
512 fiber optical rotary joint
514 fixed side of fiber optical rotary joint
516 freely rotating side of fiber optical rotary joint
520 second motor
522 pair of gears
524 pulley
526 wires on freely rotating side
528 wires on fixed side
530 translational stage
602 swept-source laser
604 optical fiber coupler
606 imaging catheter
608 reference arm
610 fiber optic coupler
612 balanced photodetector
614 waveform digitizer
618 I/O module
620 function generator
622 amplifier
624 ultrasound pulser/receiver
702 optical assembly of multimodal imaging system
704 angled reflective element of multimodal imaging system
706 first motor of multimodal imaging system
708 excitation acoustic element
710 region where excitation acoustic wave and light coincide
712 ultrasound imaging acoustic element
802 compensation arm
804 balanced photodetector in compensation arm system
806 imaging catheter in compensation arm system

The distal tip of an imaging catheter with a dual-rotation scanning mechanism is shown in FIG. 2A. Within the distal tip a motor (202) is utilized to power an optically reflective element (204), such as, as a nonlimiting example, an angled rod mirror, to relay the light from the optical assembly (206). The motor (202) with the reflective element (204) attached provides the scanning mechanism for the optical path, which can be utilized by, but not limited to, FLIM, OCT, and/or NIRF/NIRS. An acoustic element (208) is placed at the proximal end of the optical assembly (206) and can be used for the acoustic methods such as, but not limited to, excitation for OCE, detection for PAT, and/or US imaging. The distal tip of the imaging catheter is housed within a cap (210) that is optically transparent with an opening near the acoustic element (208) allowing for acoustic wave transmission. If the cap is both optically and acoustically transparent, no opening is necessary. Thus, in some embodiments, the imaging catheter may NOT have an opening to provide for acoustic wave transmission. The optical path is relayed using an optical fiber (212). The optical fiber (212) and the electrical wires (214) for the motor (210) and the acoustic element (208) are enclosed in a torque coil (216). The torque coil (216) transfers the rotational force provided at the proximal end of the imaging catheter to provide the second rotation scanning scheme. In some embodiments, the imaging catheter may comprise only one optical assembly (206) comprising at least one optical element. In other embodiments, the image catheter may comprise more than one optical assembly (206), each comprising at least one optical element. In some embodiments, each optical assembly of the more than one optical assemblies executes a different method of optical imaging. In some embodiments, the imaging catheter may comprise only one acoustic assembly comprising the acoustic element (208). In other embodiments, the image catheter may comprise more than one acoustic assembly, each comprising at least one acoustic element. In some embodiments, each acoustic assembly of the more than one acoustic assemblies executes a different method of acoustic imaging.

The same principle can also be applied in a different configuration, as shown in FIG. 2B. In such a configuration, the acoustic element (222) is placed at the distalmost position within the cap (224), and the motor (226) and the attached reflective element (228) are placed between the acoustic element (222) and the optical assembly (230).

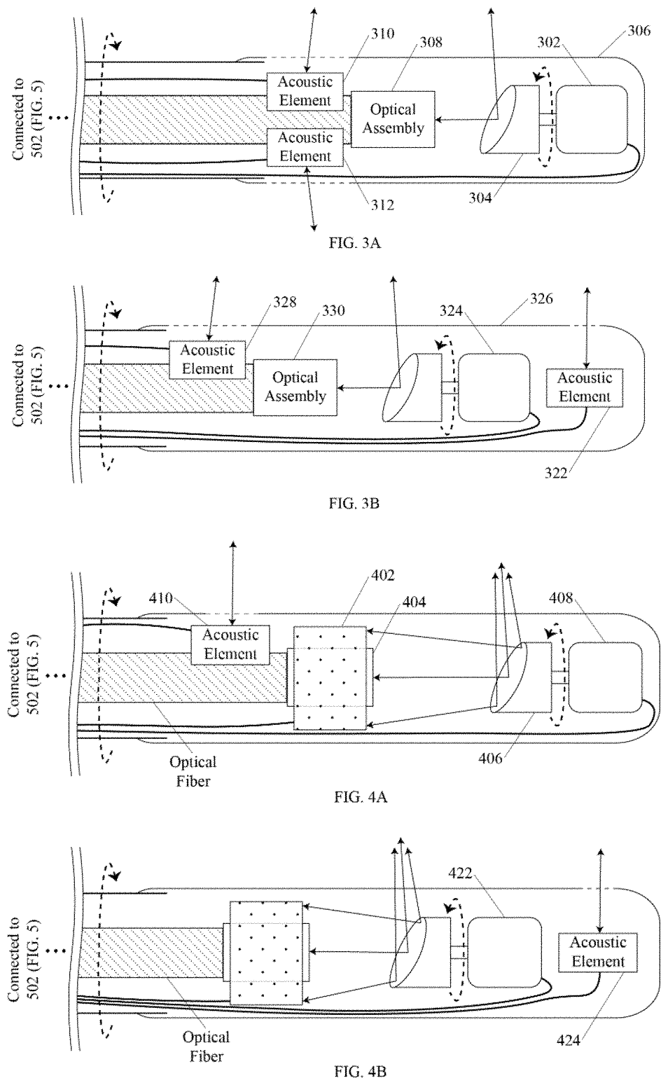

Multiple acoustic elements can be integrated within the distal tip, as depicted in FIG. 3. In FIG. 3A, the motor (302) and the attached optically reflective element (304) are placed at the distalmost cavity of the cap (306) for the optical path from the optical assembly (308) to perform rotational scanning. As a nonlimiting example herein, two acoustic elements (310), (312) are placed diametrically opposed at the proximal end of the optical assembly (308) for excitation, detection, and/or both. Two acoustic elements are shown herein, but more acoustic elements can be utilized and at various angles. Configurations that require the acoustic elements to be at different axial locations can also be realized, as shown in FIG. 3B. An acoustic element (322) can be placed distal to the motor (324), whereas another acoustic element (328) is placed proximal to the optical assembly (330).

While a typically acoustic element has a rectangular shape, circular acoustic elements with a center aperture (i.e., ring-shaped) have been developed and can also be utilized in the presented dual-rotation scanning mechanism, as well as any shape of acoustic element that provides for efficient acoustic imaging. As shown in FIG. 4A, the center aperture of the ring acoustic element (402) allows the optical assembly (404) to go through or sit within the ring acoustic element (402). In such a configuration, the alignment between the optical path from the optical element (404) and the acoustic path from the ring acoustic element (402) can be coaxial and/or confocal. In such a case, the reflective element (406) attached to the motor (408) reflects both acoustic wave and light. Another acoustic element can be placed proximal to the optical assembly (404), such as the case of the acoustic element (410) in FIG. 4A, or distal to the motor (422), such as the case of the acoustic element (424) in FIG. 4B.

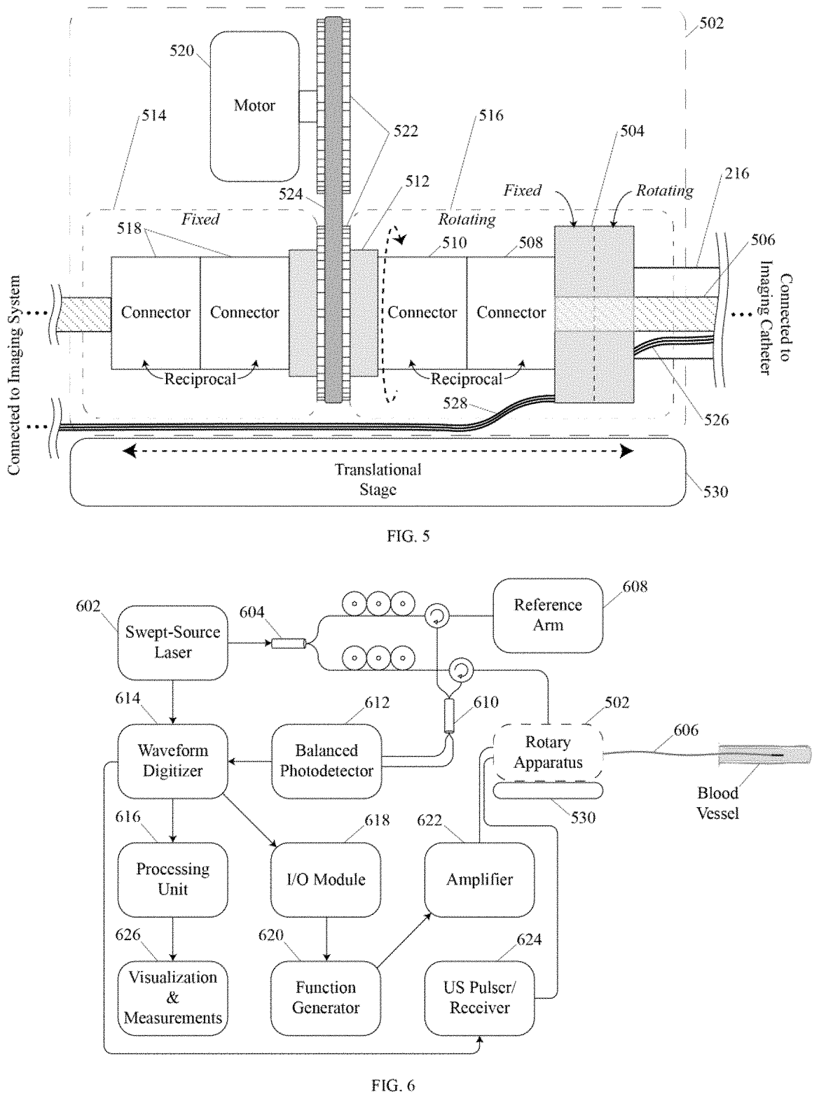

As previously described, the torque coil (216) transferring the rotational force is the second rotation scanning scheme. To provide such a rotation force, a rotary apparatus (502), as a non limiting example, depicted in FIG. 5 may be used. A hollow shaft slip ring (504) (also known as a through hole slip ring or a slip ring with through bore) allows the optical fiber (506) to pass through. The optical fiber (506) is terminated with a connector (508), which is reciprocal to the connector (510). The use of the reciprocal connectors (508), (510) allows for the imaging catheter to be detached from the rotary apparatus for replacement. The optical path goes through a fiber optical rotary joint (512) which consists of a fixed side (514) and a freely rotating side (516). The fixed side (514) has a pair of reciprocal connectors (518) that allows for the connection to an imaging system. The freely rotating side (516) is driven by a motor (520) through a pair of gears (522). In some embodiments, only one gear (522) is used to drive the freely rotating side (516). In other embodiments, more than two gears (522) are used to drive the freely rotating side (516). The gears (522) can be of various gear ratios and can be driven through a pulley (524) or direct contact. The slip ring (504) is passive, allowing for one side of the wires (526) to freely rotate with the optical path while maintaining the wires on the other side (528) fixed. This rotary apparatus (502) can be adhered to a translational stage (530), which may be motorized, for retracting the imaging catheter at a constant or known speed to generate volumetric imaging; the retraction may also be performed manually.

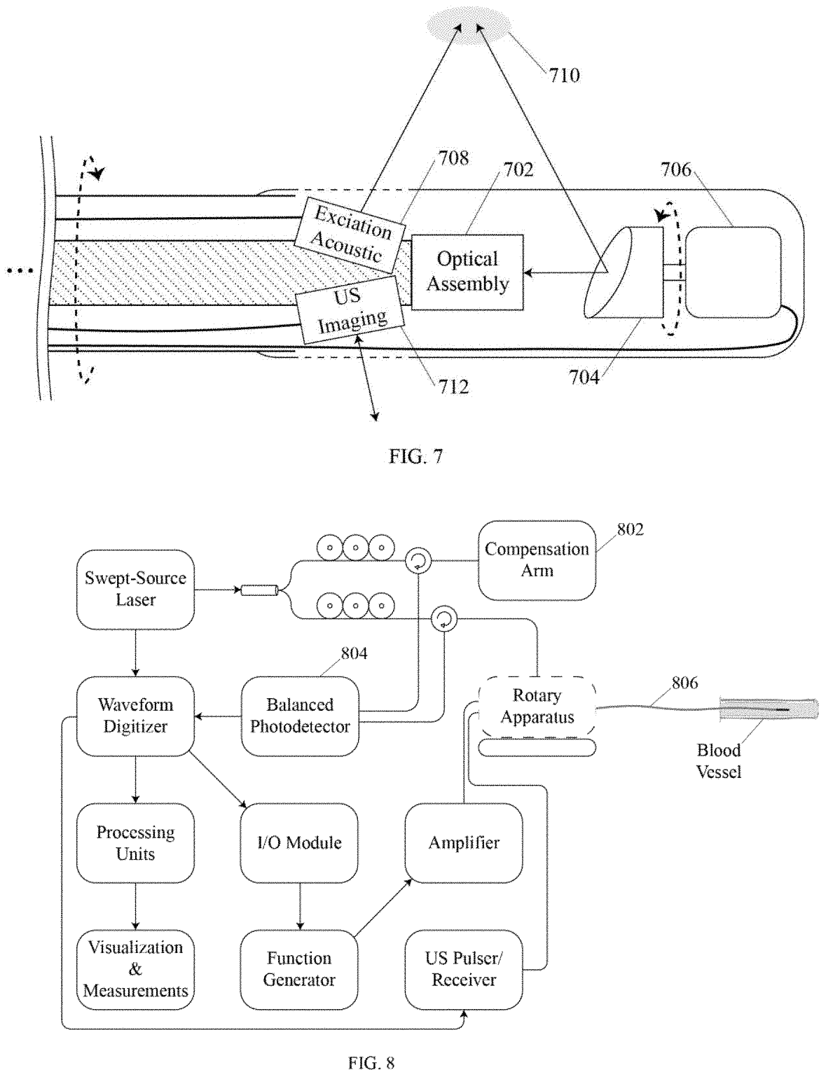

In the following, we demonstrate an application that utilizes the present dual-rotational scanning mechanism. Here, we describe a multimodal imaging system comprising OCT, OCE, and US. FIG. 6 shows the trimodality imaging system. A swept-source laser (602) is utilized as the light source of this system. In some embodiments, the swept-source laser (602) sweeps across a plurality of wavelengths. In some embodiments, the swept-source laser (602) is centered at a wavelength of 1 micron to 3 microns. The output light from the swept-source laser (602) is split by an optical fiber coupler (604), where the majority of the light is propagated to the imaging catheter (606) and the remaining light to a reference arm (608). The backscattered light from the sample collected through the imaging catheter (606) and the back-reflected light from the reference arm (608) generate the interference signal through the fiber optic coupler (610), and the interference signal is detected using a balanced photodetector (612) and digitized via a waveform digitizer (614), which is synchronized with the laser (602). A I/O module (618) is synchronized with the waveform digitizer (614) to trigger the function generator (620) to generate a waveform, which is then amplified by an amplifier (622) to drive the excitation acoustic element for OCE in the distal tip of the imaging catheter (606). An US pulser/receiver (624) is used to generate acoustic waves and receive and amplify the returning US signals for US imaging. The imaging catheter (606) is driven by the rotary apparatus (502) and retracted by the translational stage (530). The design of the imaging catheter (606) is depicted in FIG. 7. The optical assembly (702) illuminates light to the sample through an angled reflective element (704) that is driven by a motor (706) which can operate at a much higher speed compared to the rotary apparatus (502). The excitation acoustic element (708) for OCE is placed proximal to the optical assembly (702) and angled such that the excitation acoustic wave and the optical path coincide (710). The US imaging acoustic element (712) sits diametrically opposed to the excitation acoustic element (708). In such an imaging catheter configuration, OCE imaging and OCT can operate at their maximum imaging speed through the rotating reflective element (704) adhered to the motor (706) while the US and OCE excitation are performed at an appropriate imaging speed through the rotary apparatus (502).

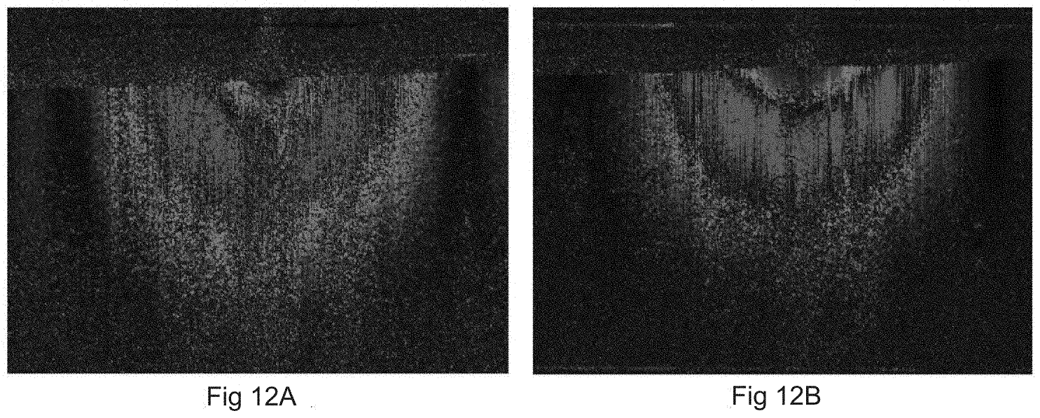

Because OCE requires high phase stability, a common-path configuration can be adapted for the optical assembly (702). In such a configuration, the distal portion of the focusing optic, typically a gradient index (GRIN) lens or GRIN fiber, is polished at an angle to reflect a small portion of the light to be used as the reference signal. The interference signal is generated from the backscattered light from the sample and the said reference signal. This imaging catheter design utilizes a modified imaging system, as described in FIG. 8. The original reference arm (608) in the previous setup is used as a compensation arm (802), whose signal is sent to one channel of the balanced photodetector (804). The interference signal from the imaging catheter (806) is fed to the other channel of the balanced photodetector (804). Such a setup offsets the undesired direct current (DC) component in the interferogram to increase the signal-to-noise ratio (SNR) and reduce phase washout effect. This is demonstrated in FIG. 12. FIGS. 12A and B present the OCE images of the same sample acquired using a reference arm (608) and a compensation arm (802), respectively. While the outward propagation of the elastic waves can be observed in both images, the result from the common-path configuration (FIG. 12B) reveals a more pronounced boundary between the upward (yellow) and downward (blue) displacement, demonstrating higher phase stability than using a reference arm (608).

Referring to FIG. 6, the present invention features a system for multimodal imaging using an imaging catheter (606). In some embodiments, the system may comprise a swept-source laser (602) that provides a light source for OCT and OCE imaging. In some embodiments, the system may further comprise a superluminescent diode, a spectrometer, and a line scan camera for OCT and OCE imaging. The system may further comprise an optical fiber coupler (604) that splits the input light source from the swept-source laser (602) into two, one for a compensation arm (802) and the other for imaging the sample using the imaging catheter (606). The system may further comprise a balanced photodetector (804) with two input channels, one taking input from the compensation arm (802) and the other from the imaging catheter (806), for offsetting DC noise. The system may further comprise a US pulser/receiver (624) for US imaging. For OCE imaging, the system may further comprise a I/O module (618) for triggering an acoustic excitation force, a function generator (620) for generating the acoustic excitation force, and an amplifier (622) for amplifying the generated acoustic excitation force. The system may further comprise a processing unit (616) for displaying and measuring results in real time, and an electronic controlling device for a first motor (706) in a distal tip of the imaging catheter (806) and a second motor (520) in a rotary apparatus (502). In some embodiments, a fiber optic coupler (610) is used for combining the signals from the compensation arm (802) and from the imaging catheter (806) prior to the delivery to the balanced photodetector (804). In some embodiments, a biased photodetector is used for detection. In other embodiments, a photomultiplier tube(s) are used for detection. In some embodiments, the first motor (706) and the second motor (520) are capable of operating at the same or different speeds, allowing for variable imaging speeds of different modalities. In other embodiments, the first motor (706) and the second motor (520) are only capable of operating at different speeds and are NOT capable of operating at different speeds. In some embodiments, a working length of the imaging catheter is at most 200 mm. In other embodiments, the working length of the imaging catheter is 100 mm to 200 mm. In other embodiments still, the working length of the imaging catheter is at least 50 mm. In other embodiments still, the working length of the imaging catheter is at least 1 mm.

The system of the present invention will perform imaging through a single imaging catheter, such that OCT, US, and OCE are performed simultaneously. This enables intrinsic image co-registration as well as reduces the overall procedure length and costs. Most importantly, it allows for a much more comprehensive analysis than single or dual modality approaches.

The system unifies the high spatial resolution of the OCT, the broad imaging depth of US, and the improved biomechanical contrast of OCE. It will provide physicians a powerful clinical instrument for studying, diagnosing, and managing vulnerable plaques. The multimodal imaging catheter only requires a single disposable guide wire and catheter, thereby reducing the costs, procedure length, associated risks, and X-ray exposure

[symbol]

The system is able to provide molecular contrast in a tissue sample for the purpose of multimodal imaging without requiring any additional preparation or alteration of the said tissue sample (i.e. applying a fluorescent pigment or dye to the tissue sample in order to aid in imaging). This is because the present invention is based on endogenous tissue chromophores, which obviates the need to alter the tissue sample to achieve efficient and accurate multimodal imaging. Note that the present invention is capable of efficiently and accurately imaging a tissue sample that has been altered by a fluorescent pigment or dye, as well as any other possible alterations, but it is not required.

### Example

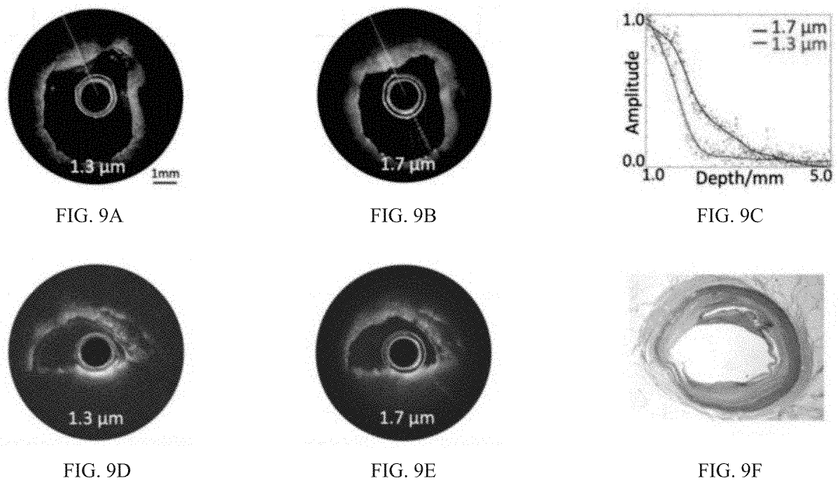

Most IVOCT systems typically utilize a 1.3-μm swept-source laser for measuring the thickness of a fibrous cap. Light in the 1.3-μm wavelength, however, cannot readily provide an adequate penetration depth for studying the deeper region of a plaque. An IVOCT system that features a 1.7-μm swept-source laser to enhance the imaging depth was proposed. Longer wavelength light has better penetration ability and can be used for visualizing information lies in the deeper layers of the vascular tissue. As shown in FIG. 9, the depth penetration performance of a 1.7- and a 1.3-μm IVOCT system by imaging cadaver coronary arteries in water are compared. Since the elastogram is a functional extension of OCT, OCE is also typically limited by the penetration depth of the light source. Therefore, as a nonlimiting example, an OCT/OCE system based on other wavelengths may be also beneficial for evaluating the biomechanical properties and structural morphology of both fibrous cap and plaque.

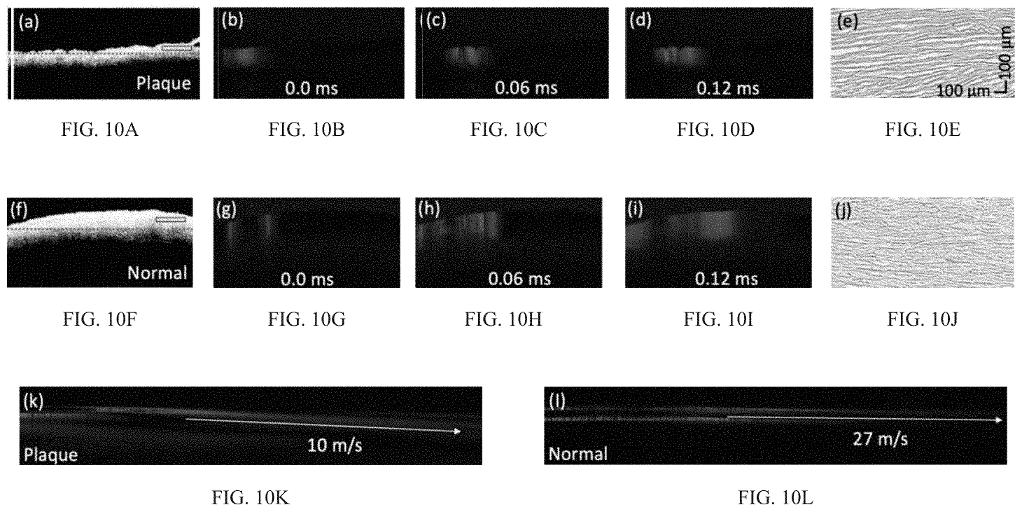

To test the feasibility of OCE for characterizing vascular tissue, a share-wave-based OCE system was implemented. In contrast to compressional-wave-based OCE, the shear-wave-based OCE provides an accurate analysis of tissue elasticity because it does not require pre-calibration or the knowledge of the applied force nor being affected by phase wrapping. Rather than calculating the Young's modulus based on localized tissue displacement, the shear-wave approach quantifies the elasticity by measuring the elastic wave velocity and is therefore invariant to phase wrapping. The system feasibility for imaging coronary arteries from a cadaver has been demonstrated, and results are shown in FIG. 10. Two representative regions of the coronary arteries, plaque and normal, are presented in FIGS. 10(A-E, K) and (F-J, L), respectively. FIGS. 10A and 10F show the OCT images of these two regions. The elastic wave propagation is depicted in FIGs (B-D) and (G-I) as a time-lapse of displacement images. Conversion to a spatiotemporal plot can determine the wave propagation speed at a given depth by calculating the slope of the wave propagation (FIGS. 10K and L). In this experiment, a decreased elasticity in the plaque was concluded.

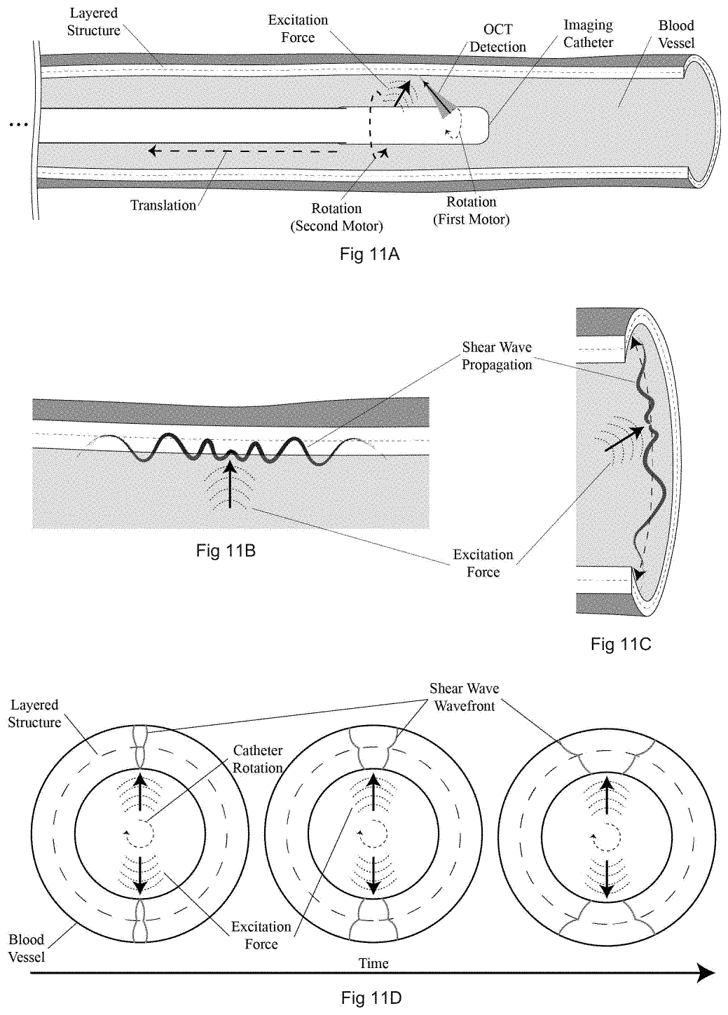

In consideration of the geometry of an arterial wall, the elastic wave in the vascular wall is assumed to be a Lamb wave. Given an excitation force (FIG. 11A), the generated shear save propagates outwards away from the excitation point and can be detected by translating (FIG. 11B) or rotating (FIG. 11C) the imaging catheter. Because a blood vessel has a multilayered structure, the generated shear wave may travel at different speeds, as illustrated in FIG. 11D wherein two excitation forces are applied at the diametrically opposed positions. OCE with optimal imaging speed is necessary to reveal these wave propagations.

The present example requires integrating OCT, US, and OCE into a singular, small form factor imaging catheter with the ability to perform ultrafast imaging for all modalities while maintaining high phase stability during scanning. FIG. 3A presents the schematic diagram of the exemplary imaging catheter. The imaging and the pushing transducer, that is, the acoustic elements (310), (312), are diametrically aligned, facing away from each other. The optical sub-probe is interposed between the two transducers. To minimize the probe dimension, a GRIN fiber is fused at the end of a single mode fiber to allow focused illumination. The emitting light from the GRIN fiber is then reflected by a rod mirror attached onto the shaft of a micromotor that can operate at 4,900 revolutions per second (rps). In order to achieve a high phase stability for accurate OCE measurement, a common-path configuration is incorporated in the optical sub-probe. This is achieved by polishing the distal end of the GRIN fiber at a specific angle to partially reflect the illumination light, which acts as the reference arm. This configuration provides a highly stable phase by minimizing the fluctuation of optical path difference between the sample and the reference arms. Furthermore, the polarization and the dispersion mismatch can be intrinsically compensated to enhance the axial resolution and SNR.

The highest IVOCT imaging speed reported is currently ˜5,600 frames per second (fps). In the conventional IVUS-OCT design where a single scanning scheme is utilized, the imaging speed of OCT is confined by that of US. Because of the slow propagation speed of acoustic waves, the imaging speed of modern IVUS is limited to ˜100 fps (30 fps in typical clinical practices). To bridge the speed gap between IVOCT and IVUS imaging, dual-rotation radial scanning is implemented, in which OCT is driven by a high-speed micromotor (distal fast scan) while US imaging and acoustic radiation force (ARF) pushing are steered through a torque coil by a rotary joint device (proximal slow scan). As such, the rotations of the optical and acoustic path can be separately controlled, achieving simultaneous but variable scanning operation. More importantly, this mechanism allows for the B-M scanning protocol because the pushing duration is substantially shorter than the period of the slow scan, such that the ARF excitation with respect to its own rotation is considered static. To achieve proximal scanning, the entire probe is rotated at 100 rps through the connection to a rotary joint device, in which an optical rotary joint with a slip ring is driven by a motor to enable rotation of the probe, as shown in FIG. 5. Since the micromotor (i.e., the first motor), which is operating at 4,900 rps, is positioned inside the tip of the imaging catheter, the rotation of the entire probe, which is 100 rps, is added to the total rotational speed, allowing the imaging catheter to acquire OCT and OCE images at 5,000 fps.

The optical and the acoustic sub-probe may be arranged in series. To achieve the desired working distance for OCE, the mirror attached to the micromotor and the pushing transducer are angled at a degree to allow for an overlapped region between the optical and the acoustic beam. This arrangement may create a longitudinal offset between the beams if the imaging range is out of the overlapped region, leading to a reduced efficiency of ARF excitation. Alternatively, a ring transducer can be implemented for a coaxial alignment between the acoustic and the optical path, as shown in FIG. 4A, where the optical and the pushing acoustic beam share the same path. A high-frequency transducer is placed proximal to the ring transducer for US imaging. This configuration allows for high excitation efficiency and maximizes the imaging range.

In addition to OCT and US, additional requirements need to be met for OCE to acquire accurate mapping of elasticity in vascular tissue in vivo. The system for the trimodal probe must have high phase stability, sufficient penetration depth, and high imaging speed. As a nonlimiting example, the schematic diagram of such an IVOCT-US-OCE is presented in FIG. 8. The system is powered by a swept-source laser. The output light from the laser source is split by a 90:10 optical fiber coupler, where 90% of the light is propagated to the sample arm (imaging catheter) and the remaining 10% to a “compensation arm” with adjustable optical power. The common-path configuration is incorporated to achieve high phase stability, in which the reference arm is accomplished by reflecting a small portion of light by the angled distal end of the GRIN fiber. The backscattered light from the sample and the back-reflected beam from the reference arm generates the interference signal, which is then delivered to one channel of the balanced photodetector. The signal from the compensation arm is collected by the other channel of the photodetector to offset the undesired DC component in the interferogram to increase the SNR and reduce phase washout effect. An US pulser/receiver is used to generate acoustic waves and receive and amplify the returning US signals for IVUS. To provide the ARF for tissue excitation, a function generator is synchronized with the trigger from the laser to generate a sine wave that is then amplified to drive the ultrasound transducer.

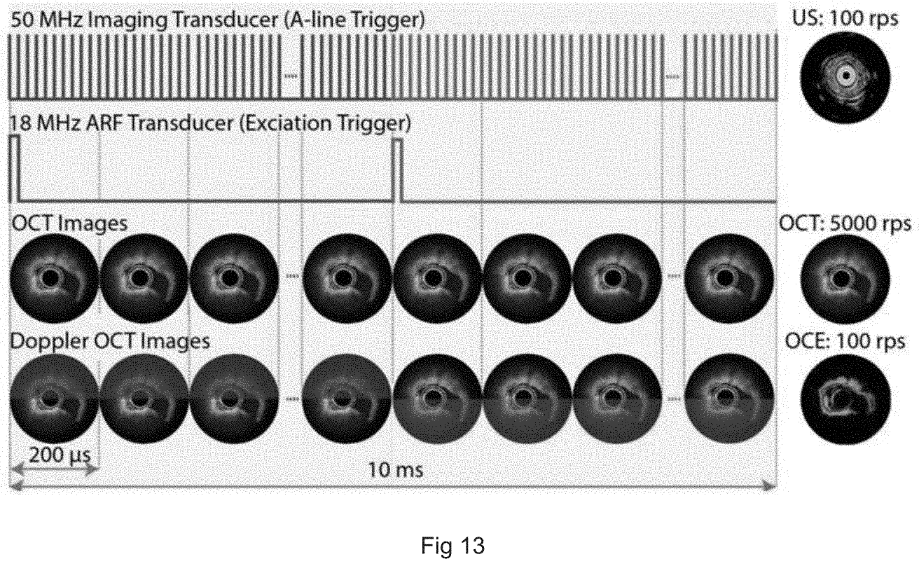

The propagation velocity of the elastic wave provides a direct measurement of the biomechanical property as pre-calibration or the knowledge of applied force is not needed to convert the displacement to elasticity. To visualize the elastic wave propagation in real time, herein a nonlimiting example of a scanning protocol is described. A B mode and B-M mode protocol are integrated for US imaging and OCT/OCE, respectively. At each longitudinal position, 1 IVUS image and 50 frames of OCT are acquired simultaneously in 10 ms. During the 50 OCT acquisition, 2 ARF excitations (duration: 20 μs) with a time interval of 5 ms are performed at the diametrically opposed positions (e.g., 0° and 180°) in order to cover entire cross-section of the vascular tissue for phase change recording. The scanning protocol is summarized in FIG. 13. Each OCT and US image consists of 800 A-line and 500 A-line, respectively. The corresponding A-line rates for OCT and US are 4 MHz and 50 kHz, respectively. Every 800 wavelength triggers (i.e., one OCT image) are down converted by a factor of 80 for US imaging synchronization (i.e., 2% of one US image). The ARF pushing transducer is excited at the 1^{st} and 20,000^{th} of OCT A trigger which is equivalent to the 1^{st} and 250^{th} of US A-line trigger.

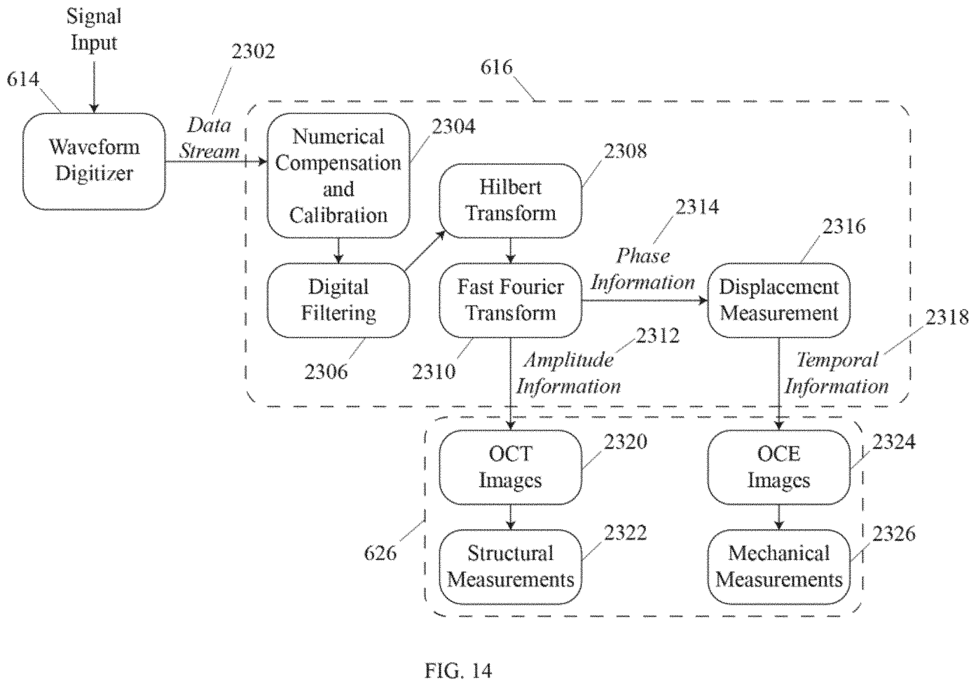

OCE data can be acquired through a phase-resolved Doppler algorithm for quantifying displacement with high sensitivity. The data processing procedure is summarized in FIG. 14. The OCT data is digitized with the calibrated k-clock and trigger to ensure linearity in the wavenumber function. As nonlimiting examples of OCT processing techniques, numerical dispersion compensation, band pass filter, etc., are first performed to optimize the axial resolution and SNR, followed by a Hilbert and a fast Fourier transform to retrieve the complex depth-encoded analytic signal containing both the amplitude and the phase terms. The OCT structural images can be reconstructed based on the amplitude information. Inter-frame analysis is performed to obtain the Doppler phase shift, Δφ, using the following equation:

$$Δ φ = [ \frac{Im ( {F}_{m} \times {F}_{m + 1}^{*} )}{Re ( {F}_{m} \times {F}_{m + 1}^{*} )} ]$$

where lm( ) and Re( ) are the imaginary and real parts of the OCT complex signal, respectively, Fₘ, is the complex signal captured at a given position, Fₘ₊₁ is Fₘ at the next time point, and F′ is the complex conjugate of F. The resulting Doppler B-scans then are resliced along the temporal direction at each depth to obtain the corresponding spatiotemporal plots. To reconstruct the 2D elastogram, the Young's moduli at each lateral position are calculated by determining the derivatives of the wave propagation in each spatiotemporal plot. Given the boundary conditions, the Young's modulus, E, can be calculated based on the Lamb wave velocity, V_{L}:

$$E = \frac{9 \times ρ \times {V}_{L}^{4}}{{( π \times f \times h )}^{2}}$$

where ρ is the vascular tissue density, f is the Lamb wave frequency, and h is the tissue thickness.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A multimodal imaging catheter utilizing a dual-rotational scanning principle for simultaneous optical and acoustic imaging to provide imagery of tissue, the imaging catheter comprising:
a. a first motor (202) in a distal tip of the imaging catheter, providing a scanning operation through a reflective element (204);
b. a rotary apparatus (502) providing a scanning operation of the imaging catheter by rotating the imaging catheter entirely, the apparatus comprising:
i. a fiber optic rotary joint (512), and
ii. a slip ring (504) driven by a second motor (520),
wherein the slip ring (504) connects to a proximal end of the imaging catheter to provide rotational force which provides an additional scanning operation;
c. an optical assembly (206) capable of focusing light for an imaging method based on optical principles; and
d. an acoustic element (208) for an imaging method based on acoustic principles;
wherein the first motor (202) and the second motor (520) can operate at the same or different speeds, allowing for variable imaging speeds of different modalities.

2. The imaging catheter of claim 1, wherein one optical assembly (206) is used for multiple imaging methods that are based on optical and/or acoustic principles.

3. The imaging catheter of claim 1, wherein multiple optical assemblies are used.

4. The imaging catheter of claim 1, wherein at least one acoustic assembly is used to generate mechanical wave in tissue.

5. The imaging catheter of claim 1, wherein one acoustic element (208) is used for multiple imaging methods that are based on optical and/or acoustic principles.

6. The imaging catheter of claim 1, wherein multiple acoustic assemblies are used.

7. The imaging catheter of claim 1, wherein at least one acoustic assembly is used for imaging, wherein at least one acoustic assembly is used to generate mechanical wave in tissue.

8. The imaging catheter of claim 1, wherein the imaging catheter is capable of performing a combination of OCT, OCE, US, PAT, and NIRS/NIRS.

9. The imaging catheter of claim 1, wherein the optical assembly (206) is configured for common-path optical assembly for high phase stability.

10. The imaging catheter of claim 1, wherein the acoustic element (208) is a ring-shaped acoustic element (402), such that an optical assembly (404) goes through or sits within the center aperture of the acoustic element (402) and the reflective element (204) is a reflective element capable of reflecting acoustic waves and light.

11. A system for multimodal imaging using a dual-rotational imaging catheter (806), the system comprising:
a. a swept-source laser (602) for providing a light source for OCT and OCE;
b. an optical fiber coupler (604) that splits the input light source from the swept-source laser (602) into two, one for a compensation arm (802) and the other for imaging the sample;
c. a balanced photodetector (804) with two input channels, one taking input from the compensation arm (802) and the other from the imaging catheter (806), for offsetting DC noise;
d. a US pulser/receiver (624) for US imaging;
e. a I/O module (618) for triggering an acoustic excitation force for OCE;
f. a function generator (620) for generating the acoustic excitation force for OCE;
g. an amplifier (622) for amplifying the generated acoustic excitation force for OCE;
h. a processing unit (616) for displaying and measurement of results in real time;
i. an electronic controlling device for a first motor (706) in a distal tip of the imaging catheter (806) and a second motor (520) in a rotary apparatus (502).

12. The system of claim 11, wherein optionally a swept-source laser (602) and a balanced photodetector (804) may be embodied in a superluminescent diode, a spectrometer, and a line scan camera are used for OCT and OCE.

13. The system of claim 12, wherein optionally a fiber optic coupler (610) is used for combining the signals from the compensation arm (802) and from the imaging catheter (806) prior the delivery to the balanced photodetector (804) for detection.

14. The system of claim 12, wherein optionally a photodetector is used for detection.

15. The system of claim 12, wherein optionally a photomultiplier tube(s) is/are used for detection.

16. The system of claim 12, wherein optionally an interference signal may be generated using backscattered light from the imaging catheter (606) and back-reflected light from the reference arm (608); wherein the detection of the interference signal is by a balanced photodetector (612).

17. The system in claim 12, wherein, a processing unit (616) is embodied for real-time visualization and measurements (626) through executing computer-readable instructions comprising:
a. Streaming data (2302) acquired by the waveform digitizer (614);
b. Performing a series of digital processing techniques, including numerical dispersion compensation and calibration for linearity (2304), digital filtering (2306), Hilbert transform (2308), and Fast Fourier transform (2310), to obtain amplitude information (2312) and phase information (2314);
c. Displaying OCT images (2320) based on the amplitude information (2312) in real time and providing structural measurements (2322);
d. Calculating displacement information (2316) based on phase information (2314);
e. With temporal information (2318), converting the displacement information (2316) to OCE images (2324) in real time, providing mechanical measurements (2326).

18. The system in claim 12, wherein optionally the digital filtering (2306) is embodied in analogue filter(s).
